(19) United States
(12) Patent Application Publication
(10) Pub. No.: US 2026/0011436 A1
(43) Pub. Date: Jan. 8, 2026

(54) MULTI-MODAL SCHEDULING FOR HEALTHCARE ORGANIZATIONS

(71) Applicant: Tiny Maple Ventures Inc., Toronto (CA)

(72) Inventor: Ryan Doherty, Toronto (CA)

(21) Appl. No.: 19/261,172

(22) Filed: Jul. 7, 2025

Related U.S. Application Data
(60) Provisional application No. 63/667,980, filed on Jul. 5, 2024.

(51) Int. Cl. G16H 40/20; G06Q 10/0631

(52) U.S. Cl. CPC G16H 40/20; G06Q 10/063116

(57) ABSTRACT

Described herein are systems and methods for allocating staffing resources across multiple operating entities. In some examples, the method includes: (a) retrieving data from a plurality of operating entities; (b) sorting the retrieved data into data groups of retrieved data subsets; (c) providing the data group with a data group identifier; (d) comparing each of the data group identifiers with stored data group identifiers of stored data groups which are stored on at least one memory device; (e) adding that retrieved data subset to one of the stored data groups or generating a new stored data group; (f) identifying a staffing resource surplus for a particular time frame; (g) determining a staffing demand for the particular time frame for at least one of the plurality of operating entities; and (h) allocating the staffing resource surplus to the operating entity having the greatest staffing demand.

### CROSS REFERENCE TO RELATED APPLICATIONS

This application claims the benefit of the filing date of U.S. Provisional Patent Application Ser. No. 63/667,980, filed Jul. 5, 2024, the specification of which is incorporated herein by reference.

### FIELD

The specification relates generally to work force scheduling, and specifically to systems and methods for creating staffing efficiencies across multiple operating entities in the healthcare field through effective allocation and movement of staffing resources, namely personnel.

### BACKGROUND

Work force scheduling is often completed with reference solely to historic practices (i.e., routine) and in a location-specific manner, disregarding the operating status and work force schedules of entities operating in the same field within the same approximate geographical location, which can lead to inefficient allocation of staff and limited access to services offered by such staff. Further, work force scheduling is often completed without consideration of factors beyond staff-shortages. That is, work force scheduling is often completed in a binary manner, in which a staffing shortage is identified and an employee is allocated, but historically, no consideration is put into which particular employee would most effectively/efficiently remedy that staffing shortage. Further, under traditional methods of work force scheduling, entities tend to rely on manual processes such as calling staff and requiring advance notice of time off for the purpose of finding replacements, which can be cumbersome and costly.

Within the field of healthcare, ineffective allocation of staffing can have a significant negative impact on budgets, delivery of services, care coordination, and access to care.

### SUMMARY OF THE INVENTION

This summary is intended to introduce the reader to the more detailed description that follows and not to limit or define any claimed or as yet unclaimed invention. One or more inventions may reside in any combination or sub-combination of the elements or method steps disclosed in any part of this document including its claims and figures.

According to one aspect of this disclosure, there is provided a method for allocating staffing resources across multiple operating entities. The method for allocating staffing resources across multiple operating entities may include retrieving data from a plurality of operating entities, wherein one or more of the plurality of operating entities may comprise at least one of structured and unstructured data. The method for allocating staffing resources across multiple operating entities may include sorting the retrieved data into data groups of retrieved data subsets, wherein the retrieved data subsets may include at least one of: (a) number of staff scheduled to work at a particular operating entity on a particular date; (b) client demand based on a utilization demand forecast; (c) qualifications and/or preferences of the staff; (d) efficiencies of the staff of the operating entity; (e) operating status of the operating entity on a particular date; (f) billing and/or demographic information of clients of the operating entities; and (g) location of the operating entity. The method for allocating staffing resources across multiple operating entities may include for each of the data groups, providing the data group with a data group identifier. The method for allocating staffing resources across multiple operating entities may include comparing each of the data group identifiers with stored data group identifiers of stored data groups which are stored on at least one memory device, wherein the stored data groups may each comprise at least one previously retrieved data subset. For each of the retrieved data subsets of each of the data groups the method for allocating staffing resources across multiple operating entities may include (a) adding that retrieved data subset to one of the stored data groups of the stored data groups if the data group identifier of the data group of that retrieved data subset matches a stored data group identifier of the stored data group, that retrieved data subset being added to the stored data group having a stored data group identifier that matches the data group identifier of the data group of that retrieved data subset; or (b) generating a new stored data group with the stored data groups which are stored on the at least one memory device, the new stored data group including that retrieved data subset, if the data group identifier of the data group of that retrieved data subset does not match a stored data group identifier of the stored data groups. The method for allocating staffing resources across multiple operating entities may include identifying a staffing resource surplus for a particular time frame. The method for allocating staffing resources across multiple operating entities may include determining a staffing demand for the particular time frame for at least one of the plurality of operating entities. The method for allocating staffing resources across multiple operating entities may include allocating the staffing resource surplus to the operating entity having the greatest staffing demand.

In some embodiments, the staffing demand of each operating entity of the at least one operating entities may be determined by comparing the number of staff scheduled to work during the particular time frame to a predicted number of staff suggested to work during the particular time frame, wherein the predicted number of staff suggested to work during the particular time frame may be based on at least one of: (a) efficiencies of the staff of the operating entity, wherein the efficiencies of the staff of the operating entity may be based on the stored data groups; (b) estimated population density of the area surrounding the operating entity, wherein the estimated population density of the area surrounding the operating entity may be based on the stored data groups; (c) historic ratio of client visits to client arrivals, wherein the historic ratio of client visits to client arrivals may be based on the stored data groups; (d) number of clients received by the operating entity that have a billing address in closer proximity to a different operating entity, wherein the number of clients received by the operating entity that have a billing address in closer proximity to a different operating entity may be based on the stored data groups; (e) client demographic context, wherein the client demographic context may be based on the stored data groups; (f) client visit context, wherein the client visit context may be based on the stored data groups; (g) utilization demand forecast, wherein the utilization demand forecast may be based on the stored data groups; and (h) attempted bookings, wherein the attempted bookings may be based on the stored data groups.

In some embodiments, the staffing demand of each operating entity of the at least one operating entities may be determined by comparing the number of staff scheduled to work during the particular time frame to a predicted number of staff suggested to work during the particular time frame, wherein the predicted number of staff suggested to work during the particular time frame may be based on at least two of (a) efficiencies of the staff of the operating entity, wherein the efficiencies of the staff of the operating entity may be based on the stored data groups; (b) estimated population density of the area surrounding the operating entity, wherein the estimated population density of the area surrounding the operating entity may be based on the stored data groups; (c) historic ratio of client visits to client arrivals, wherein the historic ratio of client visits to client arrivals may be based on the stored data groups; (d) number of clients received by the operating entity that have a billing address in closer proximity to a different operating entity, wherein the number of clients received by the operating entity that have a billing address in closer proximity to a different operating entity may be based on the stored data groups; (e) client visit context, wherein the client visit context may be based on the stored data groups; (f) utilization demand forecast, wherein the utilization demand forecast may be based on the stored data groups; and (g) attempted bookings, wherein the attempted bookings may be based on the stored data groups.

In some embodiments, the staffing demand of each operating entity of the at least one operating entities is determined by comparing the number of staff scheduled to work during the particular time frame to a predicted number of staff suggested to work during the particular time frame, wherein the predicted number of staff suggested to work during the particular time frame may be based on (a) efficiencies of the staff of the operating entity, wherein the efficiencies of the staff of the operating entity may be based on the stored data groups; (b) estimated population density of the area surrounding the operating entity, wherein the estimated population density of the area surrounding the operating entity may be based on the stored data groups; (c) historic ratio of client visits to client arrivals, wherein the historic ratio of client visits to client arrivals may be based on the stored data groups; (d) number of clients received by the operating entity that have a billing address in closer proximity to a different operating entity, wherein the number of clients received by the operating entity that have a billing address in closer proximity to a different operating entity may be based on the stored data groups; (e) client visit context, wherein the client visit context may be based on the stored data groups; (f) utilization demand forecast, wherein the utilization demand forecast may be based on the stored data groups; and (g) attempted bookings, wherein the attempted bookings may be based on the stored data groups.

In some embodiments, the staffing demand of each operating entity of the at least one operating entities is determined by: (a) comparing the number of staff scheduled to work during the particular time frame to a predicted number of staff suggested to work during the particular time frame, wherein the predicted number of staff suggested to work during the particular time frame may be based on at least one of: (i) efficiencies of the staff of the operating entity, wherein the efficiencies of the staff of the operating entity may be based on the stored data groups; (ii) estimated population density of the area surrounding the operating entity, wherein the estimated population density of the area surrounding the operating entity may be based on the stored data groups; (iii) historic ratio of client visits to client arrivals, wherein the historic ratio of client visits to client arrivals may be based on the stored data groups; (iv) number of clients received by the operating entity that have a billing address in closer proximity to a different operating entity, wherein the number of clients received by the operating entity that have a billing address in closer proximity to a different operating entity may be based on the stored data groups; (v) client visit context, wherein the client visit context may be based on the stored data groups; (vi) utilization demand forecast, wherein the utilization demand forecast may be based on the stored data groups; and (vii) attempted bookings, wherein the attempted bookings may be based on the stored data groups; and (b) identifying any dependencies of the staffing resource surplus, wherein the dependencies of the staffing resource surplus may be based on the stored data groups.

In some embodiments, the staffing resource surplus identified may be scheduled to work during the particular time frame for one of the operating entities which is not the operating entity having the greatest staffing demand.

In some embodiments, determining the staffing demand may comprise use of artificial intelligence and/or data analytics.

In some embodiments, the method may further comprise identifying a second staffing resource surplus for the particular time frame; and allocating the second staffing resource surplus to the operating entity having the second greatest staffing demand.

In some embodiments, the staffing resource surplus may be a first staffing resource surplus and the method may further comprise (a) identifying a second staffing resource surplus for the particular time frame; (b) after allocating the first staffing resource surplus to the operating entity having the greatest staffing demand, updating the staffing demand for the particular time frame for the at least one of the plurality of operating entities; and (c) after updating the staffing demand for the particular time frame for the at least one of the plurality of operating entities, allocating the second staffing resource surplus to the operating entity having the greatest staffing demand.

In some embodiments, the staffing resource surplus may be a first staffing resource surplus and the method may further comprise (a) identifying a second staffing resource surplus for the particular time frame; (b) determining a first distance between the first staffing resource surplus and the operating entity having the greatest staffing demand; and (c) determining a second distance between the second staffing resource surplus and the operating entity having the greatest staffing demand. Wherein allocating the staffing resource surplus to the operating entity having the greatest staffing demand may include (i) allocating the first staffing resource surplus to the operating entity having the greatest staffing demand if the first distance is less than the second distance; or (ii) allocating the second staffing resource surplus to the operating entity having the greatest staffing demand if the second distance is less than the first distance.

In some embodiments, data may be retrieved from each one of the plurality of operating entities at different frequencies.

In some embodiments, at least one of the plurality of operating entities may be associated with a healthcare entity.

In some embodiments, the staffing resource surplus may be a healthcare professional.

In some embodiments, data may be retrieved from the plurality of operating entities in real-time.

In some embodiments, allocating the staffing resource surplus to the operating entity having the greatest staffing demand may comprise transferring the staffing resource surplus to the operating entity having the greatest staffing demand.

In accordance with another aspect, there is provided a system for generating a data subset prediction. The system for generating a data subset prediction may comprise a data handling engine in communication with a plurality of data sources comprising (a) at least one memory device configured to store computer-executable instructions and the data subset prediction; (b) a processing device coupled to the memory device. The computer executable instructions when executed by the processing device may cause the processing device to retrieve data from a plurality of operating entities, wherein one or more of the plurality of operating entities comprises at least one of structured and unstructured data. The computer executable instructions when executed by the processing device may cause the processing device to sort the retrieved data into data groups of retrieved data subsets. The retrieved data subsets may include at least one of: (a) number of staff scheduled to work at a particular operating entity on a particular date; (b) client demand based on a utilization demand forecast; (c) qualifications and/or preferences of the staff; (d) efficiencies of the staff of the operating entity; (e) operating status of the operating entity on a particular date; (f) billing and/or demographic information of clients of the operating entities; and (g) location of the operating entity. The computer executable instructions when executed by the processing device may cause the processing device to, for each of the data groups, provide the data group with a data group identifier. The computer executable instructions when executed by the processing device may cause the processing device to compare each of the data group identifiers with stored data group identifiers of stored data groups which are stored on at least one memory device, wherein the stored data groups may each comprise at least one previously retrieved data subset. The computer executable instructions when executed by the processing device may cause the processing device to, for each of the retrieved data subsets of each of the data groups, (a) add that retrieved data subset to one of the stored data groups of the stored data groups if the data group identifier of the data group of that retrieved data subset matches a stored data group identifier of the stored data group, that retrieved data subset being added to the stored data group having a stored data group identifier that matches the data group identifier of the data group of that retrieved data subset; or (b) generate a new stored data group with the stored data groups which are stored on the at least one memory device, the new stored data group including that retrieved data subset, if the data group identifier of the data group of that retrieved data subset does not match a stored data group identifier of the stored data groups. The computer executable instructions when executed by the processing device may cause the processing device to identify a staffing resource surplus for a particular time frame. The computer executable instructions when executed by the processing device may cause the processing device to determine a staffing demand for the particular time frame for at least one of the plurality of operating entities. The computer executable instructions when executed by the processing device may cause the processing device to allocate the staffing resource surplus to the operating entity having the greatest staffing demand.

In some embodiments, the computer executable instructions, when executed by the processing device, may cause the processing device to determine the staffing demand for the particular time frame for at least one of the plurality of operating entities by: (a) comparing the number of staff scheduled to work during the particular time frame to a predicted number of staff suggested to work during the particular time frame, wherein the predicted number of staff suggested to work during the particular time frame may be based on at least one of: (i) efficiencies of the staff of the operating entity, wherein the efficiencies of the staff of the operating entity may be based on the stored data groups; (ii) estimated population density of the area surrounding the operating entity, wherein the estimated population density of the area surrounding the operating entity may be based on the stored data groups; (iii) historic ratio of client visits to client arrivals, wherein the historic ratio of client visits to client arrivals may be based on the stored data groups; (iv) number of clients received by the operating entity that have a billing address in closer proximity to a different operating entity, wherein the number of clients received by the operating entity that have a billing address in closer proximity to a different operating entity may be based on the stored data groups; (v) client visit context, wherein the client visit context may be based on the stored data groups; (vi) utilization demand forecast, wherein the utilization demand forecast may be based on the stored data groups; and (v) attempted bookings, wherein the attempted bookings may be based on the stored data groups; and (b) identifying any dependencies of the staffing resource surplus, wherein the dependencies of the staffing resource surplus may be based on the store data groups.

In some embodiments, the retrieved data may be encrypted and the computer-executable instructions when executed by the processing device may further cause the processing device to decrypt the retrieved data.

In some embodiments, the system may further comprise an application interface for the data handling engine.

In accordance with another aspect, there is provided a non-transitory computer readable medium for generating a data subset prediction. The non-transitory computer readable medium for generating a data subset prediction may comprise computer-executable instructions for retrieving data from a plurality of operating entities, wherein one or more of the plurality of operating entities comprises at least one of structured and unstructured data. The non-transitory computer readable medium for generating a data subset prediction may comprise computer-executable instructions for sorting the retrieved data into data groups of retrieved data subsets. The retrieved data subsets may include at least one of: (a) number of staff scheduled to work at a particular operating entity on a particular date; (b) client demand based on a utilization demand forecast; (c) qualifications and/or preferences of the staff; (d) efficiencies of the staff of the operating entity; (e) operating status of the operating entity on a particular date; (f) billing and/or demographic information of clients of the operating entities; and (g) location of the operating entity. The non-transitory computer readable medium for generating a data subset prediction may comprise computer-executable instructions for, for each of the data groups, providing the data group with a data group identifier. The non-transitory computer readable medium for generating a data subset prediction may comprise computer-executable instructions for comparing each of the data group identifiers with stored data group identifiers of stored data groups which are stored on at least one memory device. The stored data groups may each comprise at least one previously retrieved data subset. The non-transitory computer readable medium for generating a data subset prediction may comprise computer-executable instructions for, for each of the retrieved data subsets of each of the data groups, (a) adding that retrieved data subset to one of the stored data groups of the stored data groups if the data group identifier of the data group of that retrieved data subset matches a stored data group identifier of the stored data group, that retrieved data subset being added to the stored data group having a stored data group identifier that matches the data group identifier of the data group of that retrieved data subset; or (b) generating a new stored data group with the stored data groups which are stored on the at least one memory device, the new stored data group including that retrieved data subset, if the data group identifier of the data group of that retrieved data subset does not match a stored data group identifier of the stored data groups. The non-transitory computer readable medium for generating a data subset prediction may comprise computer-executable instructions for identifying a staffing resource surplus for a particular time frame. The non-transitory computer readable medium for generating a data subset prediction may comprise computer-executable instructions for determining a staffing demand for the particular time frame for at least one of the plurality of operating entities. The non-transitory computer readable medium for generating a data subset prediction may comprise computer-executable instructions for allocating the staffing resource surplus to the operating entity having the greatest staffing demand.

In some embodiments, the non-transitory computer readable medium may further comprising computer-executable instructions for determining the staffing demand of each operating entity of the at least one operating entities by (a) comparing the number of staff scheduled to work during the particular time frame to a predicted number of staff suggested to work during the particular time frame, wherein the predicted number of staff suggested to work during the particular time frame may be based on at least one of: (i) efficiencies of the staff of the operating entity, wherein the efficiencies of the staff of the operating entity may be based on the stored data groups; (ii) estimated population density of the area surrounding the operating entity, wherein the estimated population density of the area surrounding the operating entity may be based on the stored data groups; (iii) historic ratio of client visits to client arrivals, wherein the historic ratio of client visits to client arrivals may be based on the stored data groups; (iv) number of clients received by the operating entity that have a billing address in closer proximity to a different operating entity, wherein the number of clients received by the operating entity that have a billing address in closer proximity to a different operating entity may be based on the stored data groups; (v) client visit context, wherein the client visit context may be based on the stored data groups; (vi) utilization demand forecast, wherein the utilization demand forecast may be based on the stored data groups; and (vii) attempted bookings, wherein the attempted bookings may be based on the stored data groups; and (b) identifying any dependencies of the staffing resource surplus, wherein the dependencies of the staffing resource surplus may be based on the store data groups.

### BRIEF DESCRIPTIONS OF THE DRAWINGS

For a better understanding of the various aspects of the application described herein and to show more clearly how they may be carried into effect, reference will now be made, by way of example only, to the accompanying drawings in which:

FIG. 1 depicts a flowchart illustrating a method of allocating staffing resources across multiple operating entities, according to non-limiting embodiments; and

FIG. 2 depicts a schematic of a system for allocating staffing resources across multiple operating entities, according to non-limiting embodiments.

### DETAILED DESCRIPTION

Herein described are systems and methods for allocating staffing resources across multiple operating entities. Staffing resources can include, for example, clinical staff, direct care providers such as physicians, nurse practitioners, physician assistants, nurses, nurse practitioners, respiratory therapists, dietitians, dentists, pharmacists, speech-language pathologists, physical therapists, occupational therapists, behavioral therapists, allied health professionals, specialists, technical specialists, phlebotomists, medical laboratory scientists, social workers, support & administrative staff, patient/client registration staff, and other administrative and/or clinical personnel.

The systems and methods described herein collect and utilize data that is not commonly considered by known means of allocating staffing resources, individually or jointly, to allocate staffing resource surpluses (i.e., completing work force scheduling) to effectively remedy staffing demands/shortages.

It will be appreciated that for simplicity and clarity of illustration, where considered appropriate, reference numerals may be repeated among the figures to indicate corresponding or analogous elements. In addition, numerous specific details are set forth in order to provide a thorough understanding of the exemplary aspects of the present application described herein. However, it will be understood by those of ordinary skill in the art that the exemplary aspects described herein may be practiced without these specific details. In other instances, well-known methods, procedures and components have not been described in detail so as not to obscure the exemplary aspects described herein. Also, the description is not to be considered as limiting the scope of the exemplary aspects described herein. Any systems, method steps, components, parts of components, and the like described herein in the singular are to be interpreted as also including a description of such systems, method steps, components, parts of components, and the like in the plural, and vice versa.

When allocating staffing resources across multiple operating entities which relate to healthcare, there may be an increased desire to allocate staffing resources, namely personnel, in an efficient manner because of the severity of the repercussions that can stem from understaffed and underperforming healthcare entities, including increased patient wait times and diminished patient outcomes. Currently, there are many challenges when allocating staffing resources across multiple operating entities, particularly because individual healthcare professionals may have particular skill sets, specializations, training experience, and preferences, and may be dependent on the availability of other resources (material or human) to perform their job. It has been found that known methods for allocating staffing resources across multiple operating entities which relate to healthcare do not account for these factors. Another challenge is that scheduling is often completed by employees of the entity. When the entity is understaffed, having the already overworked staff find replacements has been found to be highly inefficient.

For example, it may be known that a hospital will be short staffed on a particular day and known that five clinicians are available to work at 3 clinical units that day. If, however, there are not enough administrative and patient/client registration staff at the hospital on that day, which are necessary to register and manage patients/clients (a human dependency), the addition of another five clinicians may not remedy the staffing shortage. To continue with this example, it may be that a different hospital, i.e., a second hospital, may have a surplus of administrative and patient/client registration staff. Using the systems and methods described herein, it may be determined that the staffing resource surplus might be best utilized by assigning some of the staff to work at the second hospital or to assign at least one of the administrative and patient/client registration staff from the second hospital (if at least one of the administrative and patient/client registration staff is considered a staffing resource surplus with respect to the second hospital) and at least one of the five clinicians to the first hospital. Staffing allocation using systems and methods currently available to multiple operating entities which relate to healthcare may not have sufficient intra-entity communication nor sufficient detail regarding staffing resources that would allow for the accounting of these details leading to efficient allocation of staffing resources.

As outlined in more detail below, there are many factors that the systems and methods described herein may take into account when allocating a staffing resource surplus to an operating entity. Further, there are many factors that the systems and methods described herein may take into account when determining which operating entity has the greatest staffing demand. Accordingly, the systems and methods described herein may use artificial intelligence and/or data analytics to process the large amount of data required to effectively allocate staffing resources.

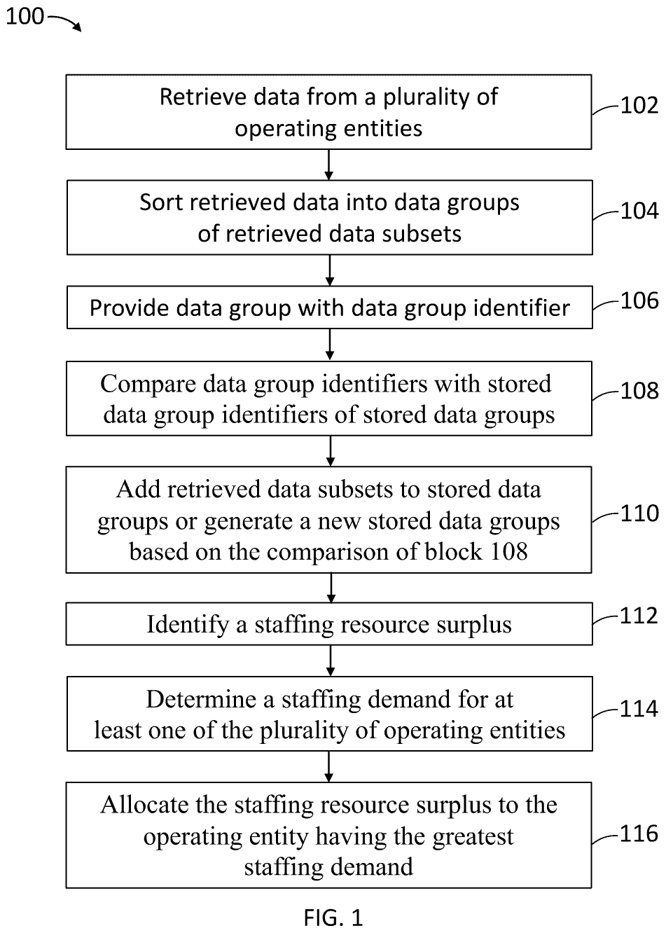

Referring now to FIG. 1, shown therein is a flowchart of a method 100 for allocating staffing resources across multiple operating entities, according to non-limiting aspects of the application. It is to be emphasized that method 100 need not be performed in the exact sequence as shown, unless otherwise indicated; and likewise various blocks may be performed in parallel rather than in sequence; hence the elements of method 100 may be referred to herein as “blocks” rather than “steps”. Further, it is to be understood that while the description herein relates generally to the healthcare industry, the systems and methods may be applied to and used in other industries as well.

As shown in FIG. 1, method 100 may start at block 102, in which data is retrieved from a plurality of data sources. The data sources may be any entity (e.g., an operating entity) capable of providing data. In some examples, the data sources are associated with healthcare operating entities (e.g., a pharmacy, hospital, medical clinic, government healthcare agency, etc.) and/or an individual (e.g., a patient/client, physician, other healthcare professionals, etc.).

In some examples, data may be retrieved from a given entity through integrations with clinical systems (e.g., electronic medical records or electronic health records), directories, portals, and processes. Alternatively, or additionally, the systems and methods described herein may be integrated with external systems to pull data from operational sources. In some examples, data may be retrieved from a given entity through physical measurement and surveying of patients and systems and corresponding input of such data by an operator.

The data sources may comprise structured and/or unstructured data. Further, the data sources may comprise a variety of data, including data that may be incongruous or otherwise conflicting with other data. The data sources may comprise any suitable data storage type or technical type. For example, according to some embodiments, the data sources comprise one or more of a database, a data feed, and a data structure.

As noted above, each of the plurality of data sources may comprise structured and/or unstructured data. Structured data for healthcare facilities can include, for example, healthcare organization name, telecommunication information, mailing address, physical location, hours of operations, scheduling information, affiliations, service availability, identity of staff members, billing information of clients, and many other facility, operational and technical attributes. Structured data for healthcare professionals can include, for example, name, date of birth, academic credentials, affiliations with professional associations, academic and professional experience, scheduling information, and many other personal and professional attributes. Unstructured data about healthcare facilities can include, for example, descriptive information about the healthcare facility, services offered, client demographics, performance efficiencies, and public discourse related to the organization and services it offers, along with many other informational attributes. Unstructured data about healthcare professionals can include, for example, preferable patient attributes (e.g., children, elderly, pregnant, from a minority group, etc.)

According to some embodiments, data may be retrieved from each one of the data sources at a different frequency. For example, according to some embodiments, data may be retrieved from a first data source on a real-time basis, whereas data may be retrieved from a second data source daily, weekly, monthly, quarterly, or annually. Accordingly, it is to be understood that at block 102, retrieving data from a plurality of data sources may or may not take place over an extended period of time. Further, the data may be retrieved from one data source in a first way and retrieved from a second data source in an alternative way (e.g., by way of electronic data transfer vs. physical collection).

Still with reference to FIG. 1, at block 104, the retrieved data is sorted into data groups of retrieved data subsets. A data group includes data (i.e., retrieved data subsets) that relates to a particular type of data. For example, a data group may be hours of operation of a first operating entity. A data group may be hours of operation of the first operating entity on a particular statutory holiday. As another example, a data group may be an indication of how many flu vaccines a second operating entity has in stock. As yet further examples, a data group may be any of (a) number of staff scheduled to work at a particular operating entity on a particular date; (b) qualifications and/or preferences of the staff (e.g., regarding preferences, if a staff member prefers to work with particular client types (e.g., infant, elderly, chronically ill, particular minority groups, etc.), work particular hours, or work at particular locations, etc.); (c) efficiencies of the staff of the operating entity (e.g., if the operating entity generally runs lean, if a staff member is known as a standout employee, etc.); (d) operating status of the operating entity on a particular date; (e) client demographics (e.g., age, health concerns, accessibility/mobility data, etc.); (f) estimated number of clients expected on a particular date; (g) billing information of clients of the operating entities; and (h) location of the operating entity.

In some examples, a data group may only have one data subset therein (one operating entity might provide data about one data type). As a particular example, if only one operating entity provides information about, for example, the number of staff scheduled to work at a particular operating entity on a particular date, then the data group may be “the number of staff scheduled to work at that particular operating entity on that particular date” and the received data subset may also be data pertaining to the number of staff scheduled to work at that particular operating entity on that particular date. Alternatively, each data group may contain multiple retrieved data subsets. That is, multiple data sources of the plurality of data sources may provide data that relates to the same data group or one data source may provide multiple data points that relate to the same data group. For example, a first operating entity may provide data that identifies an individual as a nurse and a second entity may provide data that identifies the same individual as an emergency room attendant. Therefore, in this example, a data group may be “qualifications of healthcare professional”, and the retrieved data subsets may include “nurse” and “emergency room attendant”, and optionally, may further include, for example, number of years of experience associated with each attribute (which may be used to draw inferences regarding efficiencies of that healthcare professional). Accordingly, it may be appreciated that from this data group, it may be determined that nurse may be considered as an emergency room nurse.

At block 106, each of the data groups is provided with a data group identifier. Any type of identifier may be used. For example, the identifier may be a sequence of numbers or letters, or both.

After each of the data groups have been provided with a data group identifier at block 106, at block 108, each of the data group identifiers are compared with stored data group identifiers of stored data groups. Stored data groups include historic data (e.g., previously retrieved data subsets) that has been previously retrieved from the same or different data sources (e.g., different operating entities). Stored data groups may include previously retrieved data subsets of any age. That is, the previously retrieved data subsets within the stored data groups may contain data that is years, months, weeks, days, hours, minutes, or seconds old. The stored data groups may be stored on at least one memory device (discussed in more detail below).

At block 110, if the data group identifier of the data group of a retrieved data subset matches a stored data group identifier of the stored data group, then that retrieved data subset may be added to the stored data group of the stored data group having the matching stored data group identifier. Put another way, at block 110, the stored data groups may be updated to include corresponding retrieved data subsets. For example, stored data groups may be updated to reflect new operating hours of an entity or to add additional members to a listing of employees. If the retrieved data subsets conflict with the previously retrieved data subsets of the stored data groups, the data which is more trustworthy may be so identified within the stored data group. Alternatively, the less trustworthy data may be deleted from the stored data group.

At block 110, if the data group identifier of a data group of a retrieved data subset does not match a stored data group identifier of the stored data group, then a new stored data group with the stored data groups which are stored on the at least one memory device may be generated. A new stored data group may be generated, for example, for the qualifications and/or preferences of a new healthcare professional, a new batch of schedules (i.e., number of staff scheduled to work at a particular operating entity on a particular date), estimated client demand forecasted for particular dates and/or timeframes, or information about new clients.

Still referring to FIG. 1, at block 112 of method 100, a staffing resource surplus for a particular time frame is identified. The staffing resource surplus may be a healthcare professional. A healthcare professional may be, for example, direct care practitioners such as physicians, nurse practitioners, physician assistants, nurses, respiratory therapists, dentists, pharmacists, speech-language pathologists, physical therapists, occupational therapists, physical and behavior therapists, as well as allied health professionals such as phlebotomists, medical laboratory scientists, dieticians, and social workers. The particular time frame may be any period of time that the staffing resource is able to work. This may be, for example, a twelve-hour shift, an eleven-hour shift, a ten-hour shift, a nine-hour shift, an eight-hour shift, a seven-hour shift, a six-hour shift, a five-hour shift, a four-hour shift, a three-hour shift, a two-hour shift, a one-hour shift, a thirty-minute shift, a ten-minute shift, etc. (or any length of time therebetween) on one or multiple days.

After the staffing resource surplus for the particular time frame is identified at block 112, at block 114, a staffing demand for the particular time frame for at least one of the plurality of operating entities is determined.

To determine a staffing demand, in some examples, the number of staff scheduled to work on the particular day may be compared to a predicted number of staff suggested to work on the particular day. In some examples, unstructured data about healthcare professionals schedule to work on the particular day may also be considered when determining staffing demand.

To determine the predicted number of staff suggested to work the particular time frame, any one of the following may be considered individually or jointly. One, efficiencies of the staff of the operating entity, wherein the efficiencies of the staff of the operating entity are based on the stored data groups. The efficiency of the staff of the operating entity may be used to determine the predicted number of staff suggested to work the particular time frame because it may be known that a particular staff member or group of staff members is more efficient than another. Accordingly, it may be that a first operating entity has less staff working, but because of the relatively high skill level and efficiency of those staff members, they might outperform a similar operating entity with more staff working. In this case, the similar operating entity may have a greater staffing demand. Two, estimated population density of the area surrounding the operating entity, wherein the estimated population density of the area surrounding the operating entity is based on the stored data groups. The estimated population density of the area surrounding the operating entity may be used to determine the predicted number of staff suggested to work the particular time frame because the population density may provide an indication of the number of clients an operating entity could expect to receive during the particular time frame. Three, historic ratio of client visits to client arrivals, wherein the historic ratio of client visits to client arrivals are based on the stored data groups. The historic ratio of client visits to client arrivals may be used to determine the predicted number of staff suggested to work the particular time frame because this may provide an indication of how many clients unsuccessfully attempted to receive treatment, which may be an indication of extensive wait times at the operating entity and the operating entity being understaffed. Four, the number of clients received by the operating entity that have a billing address in closer proximity to a different operating entity, wherein the number of clients received by the operating entity that have a billing address in closer proximity to a different operating entity are based on the stored data groups. The number of clients received by the operating entity that have a billing address in closer proximity to a different operating entity may be used to determine the predicted number of staff suggested to work the particular time frame because this might indicate that clients are choosing to travel further distances than necessary to receive care, which might indicate that the closer operating entity may be understaffed. Five, client visit context, wherein the client visit context is based on the stored data groups. The client visit context may be used to determine the predicted number of staff suggested to work the particular time frame because certain client visits are more resource (e.g., human resource) intensive than others. That is, it may be that a first operating entity sees only a few clients over a particular time frame, but those clients require the attention of many staff members for long periods of time or length of appointment differ based on clinician attributes. Therefore, while the client numbers may be low at this particular operating entity, there may be a high staffing demand because of the type of care the clients of that operating entity require. Six, utilization demand forecast, wherein the utilization demand forecast is based on the stored data groups. The utilization demand forecast may be used to determine the predicted number of staff suggested to work the particular time frame, where the utilization demand is an estimation of client volumes for specific dates and timeframes based on the plurality of historical information for that location, clinicians & staff working on those dates & timeframes, and recent healthcare resource utilization trends. Seven, attempted bookings, wherein the attempted bookings is based on the stored data groups. The attempted bookings may be used to determine the predicted number of staff suggested to work the particular time frame because this may provide an indication of how many clients unsuccessfully attempted to receive treatment, which may be an indication of extensive wait times at an operating entity and the operating entity being understaffed.

Optionally, when determining the staffing demand, the dependencies of the staffing resource surplus may also be considered. As noted previously, a dependency of a staffing resource surplus is a material or human resource that is required to be at the operating entity to allow the staffing resource surplus to do their job. For example, while there may be a high staffing demand at a dental office because there is a large waiting list of clients in need of fillings, it may not be desirable to allocate a dentist to that dental office if that dental office does not have the materials required to complete the procedure. Dependencies of the staffing resource surplus may be determined based on the stored data groups.

In some examples, one or more of machine learning, data modelling and artificial generative intelligence (AGI) may be used identify the stored data group(s) required to determine the staffing demand for the particular time frame.

After block 114, at block 116, the staffing resource surplus is allocated to the operating entity having the greatest staffing demand. In some examples, allocating the staffing resource surplus to the operating entity having the greatest staffing demand comprises transferring the staffing resource surplus to the operating entity having the greatest staffing demand.

In some examples, method 100 may include (a) identifying a second staffing resource surplus for the particular time frame; and (b) allocating the second staffing resource surplus to the operating entity having the second greatest staffing demand. That is, in some examples, multiple staffing resource surpluses may be identified and allocated before additional data is retrieved from the plurality of data sources.

In some examples, even after being allocated the staffing resource surplus, the operating entity having the greatest staffing demand may still have the greatest staffing demand of all the operating entities. Accordingly, in some examples, the method 100 may include (a) identifying a second staffing resource surplus for the particular time frame; (b) after allocating the first staffing resource surplus to the operating entity having the greatest staffing demand, updating the staffing demand for the particular time frame for at least one of the plurality of operating entities; and (c) after updating the staffing demand for the particular time frame for at least one of the plurality of operating entities, allocating the second staffing resource surplus to the operating entity having the greatest staffing demand (which may or may not be the same operating entity that was allocated the first staffing resource surplus).

Optionally, the staffing resource surplus identified may be already scheduled to work during the particular time frame for one of the operating entities which is not the operating entity having the greatest staffing demand. That is, from the retrieved data from the plurality of operating entities it may be determined that a staffing resource at a first entity may be more effectively used if their schedule was changed such that they worked at a different entity for the particular time frame. For example, it may be that a pharmacy may have two pharmacists scheduled to work on one day and that a neighboring hospital may not have any pharmacists scheduled to work on that day. While moving one of the two pharmacists from the pharmacy to the hospital may negatively impact the pharmacy, using the systems and methods described herein, it may be determined that the added benefit to the community may be improved if one of the two pharmacists is moved from the pharmacy to the hospital for at least that one day.

In some examples, at block 112, multiple staffing resource surpluses may be identified. When multiple staffing resource surpluses are identified, which staffing resource is allocated to which operating entity may be determined as described above (i.e., the first staffing resource surplus is allocated to the operating entity having the greatest staffing demand, the second staffing resource surplus is allocated to the operating entity have the second greatest staffing demand (which may or may not be the same operating entity to which the first staffing surplus resource was allocated), etc.). However, in some examples, it may be that two staffing resource surpluses may be equally effective at remedying the greatest staffing demand. In this case, the staffing resource surplus located closest to the operating entity having the greatest staffing demand may be allocated thereto.

Accordingly, in some examples, the method 100 may include the steps of (a) identifying a second staffing resource surplus for the particular time frame; (b) determining a first distance between the first staffing resource surplus and the operating entity having the greatest staffing demand; and (c) determining a second distance between the second staffing resource surplus and the operating entity having the greatest staffing demand. Wherein allocating the staffing resource surplus to the operating entity having the greatest staffing demand includes (i) allocating the first staffing resource surplus to the operating entity having the greatest staffing demand if the first distance is less than the second distance; or (ii) allocating the second staffing resource surplus to the operating entity having the greatest staffing demand if the second distance is less than the first distance.

In some examples, unstructured data about the staffing resource surplus may be utilized when there are multiple staffing resource surpluses available to fill multiple staffing demands. For example, a particular staffing resource surplus (e.g., healthcare professional) may have a preference to work with children and one of the staffing demands may be at a children's hospital. In this example, to optimize staffing, it may be that the staffing resource surplus having a preference for working with children is allocated to the children's hospital.

Once a staffing resource surplus has been identified, and the staffing resource has been allocated to the operating entity having the greatest demand, in some examples, the operating entity in which the staffing resource surplus has been identified is to be given notification of the staffing surplus and corresponding allocation. Upon notification, the operating entity in which the staffing resource surplus has been identified will act to transfer the staffing resource surplus to the identified operating entity having the greatest demand. In some examples, this may be the physical movement of resources from one operating entity to the next. In another example, this may include notifying a staff of their amended working position at the corresponding operating entity having the greatest demand.

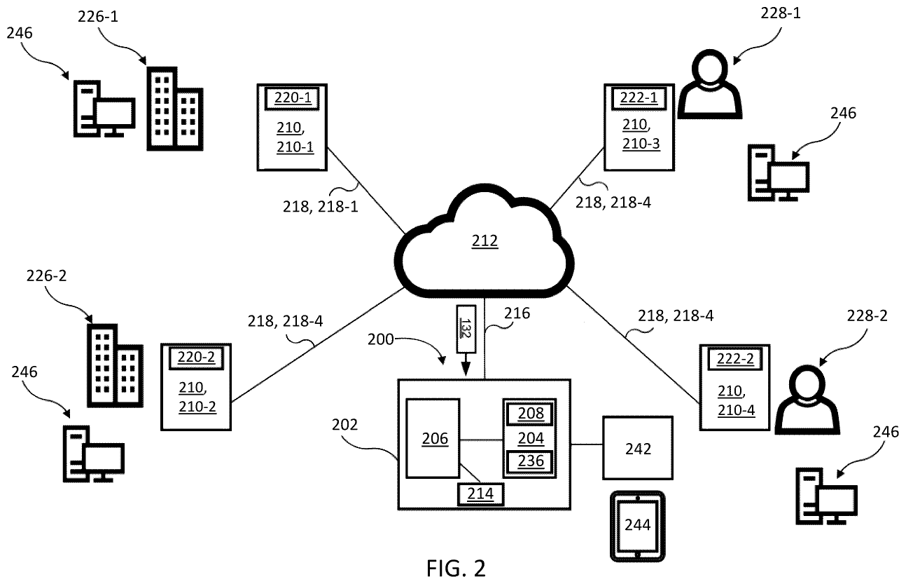

Referring now to FIG. 2, shown therein is an exemplary system 200 for generating a data subset prediction according to method 100 as described above, in accordance with non-limiting embodiments. It is to be understood, that method 100 can be implemented on variations of system 200. That is, system 200 can be varied, and need not work exactly as discussed herein, and that such variations are within the scope of present implementations.

As shown in FIG. 2, system 200 may include a data handling engine 202. The data handling engine 102 includes at least one memory 204 and at least one processing device 206. Memory 204 can comprise any suitable memory device, including but not limited to any suitable one of, or combination of, a local and/or remote volatile memory, non-volatile memory, random access memory (RAM), read-only memory (ROM), hard drive, optical drive, buffer(s), cache(s), flash memory, magnetic computer storage devices (e.g. hard disks, floppy disks, and magnetic tape), optical memory ((e.g., CD(s) and DVD(s)), and the like. Other suitable memory devices are within the scope of the application. As such, it is understood that the term “memory”, or any variation thereof, as used herein may comprise a tangible and non-transitory computer-readable medium (i.e., a medium which does not comprise only a transitory propagating signal per se) comprising or storing computer-executable instructions, such as computer programs, sets of instructions, code, software, and/or data for execution of any method(s), step(s) or process(es) described herein by any processing device(s) and/or microcontroller(s) described herein. Memory 204 comprises or is enabled to store computer-executable instructions 208 for execution by at least one processing device, including processing device 206. Memory 204 comprises or is further enabled to store stored data groups 236.

Processing device 206 is coupled to memory 204 and is enabled to control at least some of the operations system 200. As used herein, the terms “processing device”, “processing devices”, “processing device(s)”, “processor”, “processors” or “processor(s)” may refer to any combination of processing devices, and the like, suitable for carrying out the actions or methods described herein. For example, processing device 206 may comprise any suitable processing device, or combination of processing devices, including but not limited to one or multiple microprocessors, central processing units (CPUs), graphics processing units (GPUs), and the like. Other suitable processing devices are within the scope of the application.

Although system 200 is depicted as a single computing system, it is to be understood that according to some aspects of the application system 200 may comprise multiple computing systems and/or computing devices in which one or more of the computing systems and/or computing devices may be remote from each other (e.g., one or more servers, mobile devices and other suitable computing devices). Although memory 204 and processor 206 are shown as being co-located on the same computing device, it is understood that according to some embodiments memory 204 and processor 206 may be remote from each other.

Still referring to FIG. 2, in the example illustrated, system 200 is enabled to communicate with a plurality of data sources, such as data sources 210 (individually data source 210-1, data source 210-2, data source 210-3 and data source 210-4) via, for example, network 212 (which, according to some embodiments, is a secure network) to retrieve, for example, unstructured data 220-1, 220-2 and/or structured data 222-1, 222-2. For example, according to some embodiments, system 200 comprises communication module 214 coupled to processor 206. Communication module 214 may be enabled to access data sources 210 over network 212 and via, for example, communication links 216 and 218 (individually communication link 218-1, communication link 218-2, communication link 218-3, and communication link 218-4). Communication module 214 comprises any communication device(s) and/or application(s), or combination thereof, suitable for performing the communications with data sources 210 described herein. Communication links 216 and 218 comprise any suitable wired and/or wireless communication link(s), or suitable combination thereof. Communication module 214 is also enabled to communicate according to any suitable protocol which is compatible with network 212. Non-limiting examples of suitable protocols which may be compatible with network 212 are wireless protocols, cell-phone protocols, wireless data protocols, WiFi protocols, WiMax protocols, and/or a combination, or the like, such as Wired Equivalent Privacy (WEP), Wi-Fi Protected Access (WPA), Secure Sockets Layer (SSL) and Transport Layer Security (TLS). Communication module 214 may be enabled to process data for transmission between system 200 and data sources 210 in accordance to security protocols associated with network 212. For example, according to some embodiments, communication module 214 is enabled to decrypt data retrieved from any one of data sources 210 via network 212. According to some embodiments, processing device 206 is enabled similarly to communication module 214 such that processing device 206 performs at least some of the communications with computing device 110 described herein rather than communication module 214.

As described above, according to some embodiments, one or more of the data sources 210 may be associated with an entity or individual that maintains the respective one of data sources 210, such as entities 226-1, 226-2 and individuals 228-1, 228-2.

Computer-executable instructions 208, when executed by processor 206, is enabled to cause processor 206 to perform at least some portions of the method 100, as described above.

It is appreciated that, in some aspects, method 200 is implemented by system 100 by processing device 206. Indeed, method 100 is one way in which system 200 may be configured.

According to some embodiments, at least some of retrieved data is encrypted in transit and encrypted at rest to ensure secure handling of sensitive information, and to reduce data leakage.

According to some embodiments, system 200 further comprises application programming interface (API) 242 through which the functionalities and/or outputs of data handling engine 202 can be accessed. For example, according to some embodiments, API 242 is configured to provide a navigation service which may communicate with internal and external applications such as, without limitation, patient/client portals, digital front door, wayfinding websites, wayfinding applications, eReferral programs, eConsult programs, care coordination programs, call centres, ride-sharing platforms, public transit services, clinical services providers, service providers at various governmental levels (such as at the municipal, regional and national-level) and service providers at the institutional level (e.g., retail chains, banners, professional groups). According to some embodiments, API 242 provides backend access for authorized users to data handling engine 202. For example, according to some embodiments, API 242 provides access to AI prompts and/or models to enable modification of same.

API 242 may be accessed by a user through one or more computing devices, such as computing device 244. Computing device 244 comprises any suitable computing device, including but not limited to one or more portable electronic devices, mobile computing devices, portable computing devices, tablet computing devices, laptop computing devices, PDAs (personal digital assistants), cellphones, smartphones, computer terminals and the like. Other suitable computing devices are within the scope of the application. For the sake of simplicity, a single computing device 244 is shown in FIG. 2. However, according to some aspects, more than one computing device 244 is enabled to access API 242.

According to some embodiments, other functionalities of data handling engine 202, such as the outputs, may be accessed via computing devices 246 accessible by individuals 228 and/or entities 226. Computing devices 246 comprise any suitable computing devices, including but not limited to one or more portable electronic devices, mobile computing devices, portable computing devices, tablet computing devices, laptop computing devices, PDAs (personal digital assistants), cellphones, smartphones, computer terminals and the like. Other suitable computing devices are within the scope of the application.

Those skilled in the art will appreciate that in some implementations, the functionality of system 200 and method 100 can be implemented using pre-programmed hardware or firmware elements (e.g., application specific integrated circuits (ASICs), electrically erasable programmable read-only memories (EEPROMs), etc.), or other related components. In other implementations, the functionality of system 200 and method 100 can be achieved using a computing apparatus that has access to a code memory (not shown) which stores computer-readable program code for operation of the computing apparatus. The computer-readable program code could be stored on a computer readable storage medium which is fixed, tangible and readable directly by these components, (e.g., removable diskette, CD-ROM, ROM, fixed disk, USB drive). Furthermore, it is appreciated that the computer-readable program can be stored as a computer program product comprising a computer usable medium. Further, a persistent storage device can comprise the computer readable program code. It is yet further appreciated that the computer-readable program code and/or computer usable medium can comprise a non-transitory computer-readable program code and/or non-transitory computer usable medium. Alternatively, the computer-readable program code could be stored remotely but transmittable to these components via a modem or other interface device connected to a network (including, without limitation, the Internet) over a transmission medium. The transmission medium can be either a non-mobile medium (e.g., optical and/or digital and/or analog communications lines) or a mobile medium (e.g., microwave, infrared, free-space optical or other transmission schemes) or a combination thereof.

Persons skilled in the art will appreciate that there are yet more alternative aspects and modifications possible, and that the above examples are only illustrations of one or more aspects of the application. The scope, therefore, is only to be limited by the claims appended hereto.

### INTERPRETATION

It will also be understood that for the purposes of this application, “at least one of X, Y, and Z” or “one or more of X, Y, and Z” language can be construed as X only, Y only, Z only, or any combination of two or more items X, Y, and Z (e.g., XYZ, XYY, YZ, ZZ).

In the present application, components may be described as “configured to” or “enabled to” perform one or more functions. Generally, it is understood that a component that is configured to or enabled to perform a function is configured to or enabled to perform the function, or is suitable for performing the function, or is adapted to perform the function, or is operable to perform the function, or is otherwise capable of performing the function.

Additionally, components in the present application may be described as “operatively connected to”, “operatively coupled to”, and the like, to other components. It is understood that such components are connected or coupled to each other in a manner to perform a certain function. It is also understood that “connections”, “coupling” and the like, as recited in the present application include direct and indirect connections between components.

References in the application to “one embodiment”, “an embodiment”, “an implementation”, “a variant”, etc., indicate that the embodiment, implementation or variant described may include a particular aspect, feature, structure, or characteristic, but not every embodiment, implementation or variant necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely”, “only”, and the like, in connection with the recitation of claim elements or use of a “negative” limitation. The terms “preferably”, “preferred”, “prefer”, “optionally”, “may”, and similar terms are used to indicate that an item, condition or step being referred to is an optional (not required) feature of the invention.

The singular forms “a”, “an”, and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

What is claimed is:

1. A method for allocating staffing resources across multiple operating entities, comprising:
retrieving data from a plurality of operating entities, wherein one or more of the plurality of operating entities comprises at least one of structured and unstructured data;
sorting the retrieved data into data groups of retrieved data subsets, wherein the retrieved data subsets include at least one of:
(a) number of staff scheduled to work at a particular operating entity on a particular date;
(b) client demand based on a utilization demand forecast;
(c) qualifications and/or preferences of the staff;
(d) efficiencies of the staff of the operating entity;
(e) operating status of the operating entity on a particular date;
(f) billing and/or demographic information of clients of the operating entities; and
(g) location of the operating entity;
for each of the data groups, providing the data group with a data group identifier;
comparing each of the data group identifiers with stored data group identifiers of stored data groups which are stored on at least one memory device, wherein the stored data groups each comprise at least one previously retrieved data subset;
for each of the retrieved data subsets of each of the data groups,
adding that retrieved data subset to one of the stored data groups of the stored data groups if the data group identifier of the data group of that retrieved data subset matches a stored data group identifier of the stored data group, that retrieved data subset being added to the stored data group having a stored data group identifier that matches the data group identifier of the data group of that retrieved data subset; or
generating a new stored data group with the stored data groups which are stored on the at least one memory device, the new stored data group including that retrieved data subset, if the data group identifier of the data group of that retrieved data subset does not match a stored data group identifier of the stored data groups; and
identifying a staffing resource surplus for a particular time frame;
determining a staffing demand for the particular time frame for at least one of the plurality of operating entities; and
allocating the staffing resource surplus to the operating entity having the greatest staffing demand.

2. The method of claim 1, wherein the staffing demand of each operating entity of the at least one operating entities is determined by:
(a) comparing the number of staff scheduled to work during the particular time frame to a predicted number of staff suggested to work during the particular time frame, wherein the predicted number of staff suggested to work during the particular time frame is based on at least one of:
(a) efficiencies of the staff of the operating entity, wherein the efficiencies of the staff of the operating entity are based on the stored data groups;
(b) estimated population density of the area surrounding the operating entity, wherein the estimated population density of the area surrounding the operating entity is based on the stored data groups;
(c) historic ratio of client visits to client arrivals, wherein the historic ratio of client visits to client arrivals are based on the stored data groups;
(d) number of clients received by the operating entity that have a billing address in closer proximity to a different operating entity, wherein the number of clients received by the operating entity that have a billing address in closer proximity to a different operating entity are based on the stored data groups;
(e) client demographic context, wherein the client demographic context is based on the stored data groups;
(f) client visit context, wherein the client visit context is based on the stored data groups;
(g) utilization demand forecast, wherein the utilization demand forecast is based on the stored data groups; and
(h) attempted bookings, wherein the attempted bookings is based on the stored data groups.

3. The method of claim 1, wherein the staffing demand of each operating entity of the at least one operating entities is determined by:
(a) comparing the number of staff scheduled to work during the particular time frame to a predicted number of staff suggested to work during the particular time frame, wherein the predicted number of staff suggested to work during the particular time frame is based on at least two of:
(a) efficiencies of the staff of the operating entity, wherein the efficiencies of the staff of the operating entity are based on the stored data groups;
(b) estimated population density of the area surrounding the operating entity, wherein the estimated population density of the area surrounding the operating entity is based on the stored data groups;
(c) historic ratio of client visits to client arrivals, wherein the historic ratio of client visits to client arrivals are based on the stored data groups;
(d) number of clients received by the operating entity that have a billing address in closer proximity to a different operating entity, wherein the number of clients received by the operating entity that have a billing address in closer proximity to a different operating entity are based on the stored data groups;
(e) client visit context, wherein the client visit context is based on the stored data groups;
(f) utilization demand forecast, wherein the utilization demand forecast is based on the stored data groups; and
(g) attempted bookings, wherein the attempted bookings is based on the stored data groups.

4. The method of claim 2, wherein the staffing demand of each operating entity of the at least one operating entities is determined by:
(a) comparing the number of staff scheduled to work during the particular time frame to a predicted number of staff suggested to work during the particular time frame, wherein the predicted number of staff suggested to work during the particular time frame is based on:
(a) efficiencies of the staff of the operating entity, wherein the efficiencies of the staff of the operating entity are based on the stored data groups;
(b) estimated population density of the area surrounding the operating entity, wherein the estimated population density of the area surrounding the operating entity is based on the stored data groups;
(c) historic ratio of client visits to client arrivals, wherein the historic ratio of client visits to client arrivals are based on the stored data groups;
(d) number of clients received by the operating entity that have a billing address in closer proximity to a different operating entity, wherein the number of clients received by the operating entity that have a billing address in closer proximity to a different operating entity are based on the stored data groups;
(e) client visit context, wherein the client visit context is based on the stored data groups;
(f) utilization demand forecast, wherein the utilization demand forecast is based on the stored data groups; and
(g) attempted bookings, wherein the attempted bookings is based on the stored data groups.

5. The method of claim 2, wherein the staffing demand of each operating entity of the at least one operating entities is determined by:
(a) comparing the number of staff scheduled to work during the particular time frame to a predicted number of staff suggested to work during the particular time frame, wherein the predicted number of staff suggested to work during the particular time frame is based on at least one of:
(a) efficiencies of the staff of the operating entity, wherein the efficiencies of the staff of the operating entity are based on the stored data groups;
(b) estimated population density of the area surrounding the operating entity, wherein the estimated population density of the area surrounding the operating entity is based on the stored data groups;
(c) historic ratio of client visits to client arrivals, wherein the historic ratio of client visits to client arrivals are based on the stored data groups;
(d) number of clients received by the operating entity that have a billing address in closer proximity to a different operating entity, wherein the number of clients received by the operating entity that have a billing address in closer proximity to a different operating entity are based on the stored data groups;
(e) client visit context, wherein the client visit context is based on the stored data groups;
(f) utilization demand forecast, wherein the utilization demand forecast is based on the stored data groups; and
(g) attempted bookings, wherein the attempted bookings is based on the stored data groups; and
(b) identifying any dependencies of the staffing resource surplus, wherein the dependencies of the staffing resource surplus are based on the stored data groups.

6. The method of claim 1, wherein the staffing resource surplus identified is scheduled to work during the particular time frame for one of the operating entities which is not the operating entity having the greatest staffing demand.

7. The method of claim 1, wherein determining the staffing demand comprises use of artificial intelligence and/or data analytics.

8. The method of claim 1, further comprising:
identifying a second staffing resource surplus for the particular time frame; and
allocating the second staffing resource surplus to the operating entity having the second greatest staffing demand.

9. The method of claim 1, wherein the staffing resource surplus is a first staffing resource surplus and the method further comprises:
identifying a second staffing resource surplus for the particular time frame;
after allocating the first staffing resource surplus to the operating entity having the greatest staffing demand, updating the staffing demand for the particular time frame for the at least one of the plurality of operating entities; and
after updating the staffing demand for the particular time frame for the at least one of the plurality of operating entities, allocating the second staffing resource surplus to the operating entity having the greatest staffing demand.

10. The method of claim 1, wherein the staffing resource surplus is a first staffing resource surplus and the method further comprises:
identifying a second staffing resource surplus for the particular time frame;
determining a first distance between the first staffing resource surplus and the operating entity having the greatest staffing demand;
determining a second distance between the second staffing resource surplus and the operating entity having the greatest staffing demand; and
wherein allocating the staffing resource surplus to the operating entity having the greatest staffing demand includes:
allocating the first staffing resource surplus to the operating entity having the greatest staffing demand if the first distance is less than the second distance; or
allocating the second staffing resource surplus to the operating entity having the greatest staffing demand if the second distance is less than the first distance.

11. The method of claim 1, wherein data is retrieved from each one of the plurality of operating entities at different frequencies.

12. The method of claim 1, wherein at least one of the plurality of operating entities is associated with a healthcare entity and the staffing resource surplus is a healthcare professional.

13. The method of claim 1, wherein data is retrieved from the plurality of operating entities in real-time.

14. The method of claim 1, wherein allocating the staffing resource surplus to the operating entity having the greatest staffing demand comprises transferring the staffing resource surplus to the operating entity having the greatest staffing demand.

15. A system for generating a data subset prediction comprising:
a data handling engine in communication with a plurality of data sources comprising:
at least one memory device configured to store computer-executable instructions and the data subset prediction; and
a processing device coupled to the memory device;
wherein the computer executable instructions when executed by the processing device causes the processing device to:
retrieve data from a plurality of operating entities, wherein one or more of the plurality of operating entities comprises at least one of structured and unstructured data;
sort the retrieved data into data groups of retrieved data subsets, wherein the retrieved data subsets include at least one of:
(a) number of staff scheduled to work at a particular operating entity on a particular date;
(b) client demand based on a utilization demand forecast;
(c) qualifications and/or preferences of the staff;
(d) efficiencies of the staff of the operating entity;
(e) operating status of the operating entity on a particular date;
(f) billing and/or demographic information of clients of the operating entities; and
(g) location of the operating entity;
for each of the data groups, provide the data group with a data group identifier;
compare each of the data group identifiers with stored data group identifiers of stored data groups which are stored on at least one memory device, wherein the stored data groups each comprise at least one previously retrieved data subset;
for each of the retrieved data subsets of each of the data groups,
add that retrieved data subset to one of the stored data groups of the stored data groups if the data group identifier of the data group of that retrieved data subset matches a stored data group identifier of the stored data group, that retrieved data subset being added to the stored data group having a stored data group identifier that matches the data group identifier of the data group of that retrieved data subset; or
generate a new stored data group with the stored data groups which are stored on the at least one memory device, the new stored data group including that retrieved data subset, if the data group identifier of the data group of that retrieved data subset does not match a stored data group identifier of the stored data groups;
identify a staffing resource surplus for a particular time frame;
determine a staffing demand for the particular time frame for at least one of the plurality of operating entities; and
allocate the staffing resource surplus to the operating entity having the greatest staffing demand.

16. The system of claim 15, wherein the computer executable instructions, when executed by the processing device, causes the processing device to determine the staffing demand for the particular time frame for at least one of the plurality of operating entities by:
(a) comparing the number of staff scheduled to work during the particular time frame to a predicted number of staff suggested to work during the particular time frame, wherein the predicted number of staff suggested to work during the particular time frame is based on at least one of:
(a) efficiencies of the staff of the operating entity, wherein the efficiencies of the staff of the operating entity are based on the stored data groups;
(b) estimated population density of the area surrounding the operating entity, wherein the estimated population density of the area surrounding the operating entity is based on the stored data groups;
(c) historic ratio of client visits to client arrivals, wherein the historic ratio of client visits to client arrivals are based on the stored data groups;
(d) number of clients received by the operating entity that have a billing address in closer proximity to a different operating entity, wherein the number of clients received by the operating entity that have a billing address in closer proximity to a different operating entity are based on the stored data groups;
(e) client visit context, wherein the client visit context is based on the stored data groups;
(f) utilization demand forecast, wherein the utilization demand forecast is based on the stored data groups; and
(g) attempted bookings, wherein the attempted bookings is based on the stored data groups; and
(b) identifying any dependencies of the staffing resource surplus, wherein the dependencies of the staffing resource surplus are based on the store data groups.

17. The system of claim 15, wherein the retrieved data is encrypted and the computer-executable instructions when executed by the processing device further causes the processing device to decrypt the retrieved data.

18. The system of claim 15, further comprising an application interface for the data handling engine.

19. A non-transitory computer readable medium for generating a data subset prediction, comprising computer-executable instructions for:
retrieving data from a plurality of operating entities, wherein one or more of the plurality of operating entities comprises at least one of structured and unstructured data;
sorting the retrieved data into data groups of retrieved data subsets, wherein the retrieved data subsets include at least one of:
(a) number of staff scheduled to work at a particular operating entity on a particular date;
(b) client demand based on a utilization demand forecast;
(c) qualifications and/or preferences of the staff;
(d) efficiencies of the staff of the operating entity;
(e) operating status of the operating entity on a particular date;
(f) billing and/or demographic information of clients of the operating entities; and
(g) location of the operating entity;
for each of the data groups, providing the data group with a data group identifier;
comparing each of the data group identifiers with stored data group identifiers of stored data groups which are stored on at least one memory device, wherein the stored data groups each comprise at least one previously retrieved data subset;
for each of the retrieved data subsets of each of the data groups,
adding that retrieved data subset to one of the stored data groups of the stored data groups if the data group identifier of the data group of that retrieved data subset matches a stored data group identifier of the stored data group, that retrieved data subset being added to the stored data group having a stored data group identifier that matches the data group identifier of the data group of that retrieved data subset; or
generating a new stored data group with the stored data groups which are stored on the at least one memory device, the new stored data group including that retrieved data subset, if the data group identifier of the data group of that retrieved data subset does not match a stored data group identifier of the stored data groups; and
identifying a staffing resource surplus for a particular time frame;
determining a staffing demand for the particular time frame for at least one of the plurality of operating entities; and
allocating the staffing resource surplus to the operating entity having the greatest staffing demand.

20. The non-transitory computer readable medium of claim 19 further comprising computer-executable instructions for determining the staffing demand of each operating entity of the at least one operating entities by:
(a) comparing the number of staff scheduled to work during the particular time frame to a predicted number of staff suggested to work during the particular time frame, wherein the predicted number of staff suggested to work during the particular time frame is based on at least one of:
(a) efficiencies of the staff of the operating entity, wherein the efficiencies of the staff of the operating entity are based on the stored data groups;
(b) estimated population density of the area surrounding the operating entity, wherein the estimated population density of the area surrounding the operating entity is based on the stored data groups;
(c) historic ratio of client visits to client arrivals, wherein the historic ratio of client visits to client arrivals are based on the stored data groups;
(d) number of clients received by the operating entity that have a billing address in closer proximity to a different operating entity, wherein the number of clients received by the operating entity that have a billing address in closer proximity to a different operating entity are based on the stored data groups;
(e) client visit context, wherein the client visit context is based on the stored data groups;
(f) utilization demand forecast, wherein the utilization demand forecast is based on the stored data groups; and
(g) attempted bookings, wherein the attempted bookings is based on the stored data groups; and
(b) identifying any dependencies of the staffing resource surplus, wherein the dependencies of the staffing resource surplus are based on the store data groups.
